(19) United States
(12) Patent Application Publication
(10) Pub. No.: US 2006/0271018 A1
(43) Pub. Date: Nov. 30, 2006

(54) Wound drainage device

(76) Inventor: Jacob Korf, Broek Op Langedijk (NL)

Correspondence Address: TROUTMAN SANDERS LLP, 600 PEACHTREE STREET , NE, ATLANTA, GA, 30308, US

(21) Appl. No.: 10/571,873

(22) PCT Filed: Aug. 11, 2004
(86) PCT No.: PCT/NL04/00565

§ 371 Date: Jul. 27, 2006

(30) Foreign Application Priority Data
Sep. 17, 2003 (NL) 1024315
Apr. 13, 2004 (NL) 1025938

(51) Int. Cl. A61M 27/00

(52) U.S. Cl. 604/541

(57) ABSTRACT

In a wound drainage device (10) for discharging exudate originating from a wound with the aid of reduced pressure, the vacuum is created indirectly in a collection container. The collection container (16) is arranged in a vacuum chamber (14), with the space (32) between collection container (16) and vacuum chamber (14) being in communication with gas-transformer means

The invention relates to a wound drainage device for using reduced pressure to discharge exudate originating from a wound, which device comprises a housing which comprises a vacuum chamber for receiving a collection container with a feed opening for collecting exudate, and means for generating a reduced pressure in the space between the wall of the vacuum chamber and a collection container which is accommodated therein during operation.

A wound drainage device of this type is known from DE-A-21 27 764. This known system comprises a pneumatic suction device for sucking exudate out of a wound. The device comprises a housing in which there is a chamber which can be placed under a reduced pressure. A disposable flexible pouch with a suction hose for coupling to the drain which is to be positioned in or on a wound is arranged in this chamber as a collection container. The means for generating reduced pressure comprise a gas-jet pump which on one side is connected to an exchangeable gas cylinder which is likewise accommodated in the housing and on the other side is connected to the space between the said vacuum chamber and the collection container. The intention of this known wound drainage device is, inter alia, to increase its ease of use for operating staff, even at locations which are not readily accessible in emergencies.

However, it has been found that this device is relatively heavy on account of the presence of the gas cylinder and the gas-jet pump. This impedes patient mobility and has an adverse effect on its ease of handling. The gas cylinder is the only gas source for generating reduced pressure, and consequently has to be replaced frequently.

Furthermore, U.S. Pat. No. 4,004,590 has disclosed a wound drainage device in which the reduced pressure can be generated in various ways. To this end, this known device comprises firstly a suction pump with motor and secondly a connection for coupling to an external vacuum source. In this known device too, the weight of the pump and motor is a drawback to handling and therefore to patient mobility.

In this known device, as in many other known systems, use is made of electricity, in some form or another, to drive a vacuum pump. The use of electricity in a wet environment is detrimental to the safety of the wound drainage system and therefore of the patient who is to be treated.

A wound drainage device of a similar type is also known from DE-U-29 619 523. This known system comprises a vacuum-type vessel with cover. In this vessel there is a receiving pouch for exudate, which is connected to a drain via a suction line passing through a hole in the cover. The creation of reduced pressure in the vessel causes a suction force to be exerted on the wound via the receiving pouch which drains fluid from the wound. This reduced pressure in the vessel can be generated via a piston/cylinder assembly which is connected to the space between the vessel and the receiving pouch. The cover, suction line and receiving pouch form an exchangeable and sterile unit. In this known wound drainage device, the pressure prevailing in the vessel can be indicated by a spring which is accommodated in a cylinder which, via a small hole, is in communication with the interior of the vessel.

One drawback of this known system is that generation of the required vacuum in the vessel requires force to be exerted by a person in order to operate the piston/cylinder assembly, and in many cases this force cannot be provided by the patient himself. This requires the deployment of nursing staff. This manually actuated wound drainage device is generally only suitable for low-pressure applications.

It is an object of the present invention to provide a portable wound drainage device which provides patients with a high degree of freedom of movement.

A further object of the invention is to provide a wound drainage device which is very easy to use both for the patient and operating staff.

Yet another object of the invention is to provide a safe wound drainage device, which makes little or no use of electricity.

Yet a further object of the invention is to provide a wound drainage device, which can be manufactured in an easy manner.

In the wound drainage device according to the invention, for this purpose the said means for generating a reduced pressure comprise gas-transformer means for using pressurized gas to form a reduced pressure, which means on the pressure side are in communication with a pressure-resistant gas compartment and on the vacuum side are in communication with the vacuum chamber, the gas compartment being provided in the housing and having a coupling for connection to an external gas source.

In the device according to the invention, a pressure-resistant gas compartment is provided in the housing itself and can be filled with gas originating from an external gas source, such as compressed air, via the said coupling. This gas compartment is also in communication, via the gas-transformer means, with the space between the inner wall of the vacuum chamber and the outer wall of the collection container. This makes the device according to the invention “rechargeable” or “refillable” without the gas compartment itself having to be exchanged. Simple coupling to a central compressed-air line system, which in a hospital and many other care institutions has one or more connection points in virtually every ward and/or room, is easy, so that the mobile patient can carry this out himself if necessary. The device can also be used in a domestic situation if the wound drainage device is connected to the pressure side of a compressor in order to fill the gas compartment. Since there is no relatively heavy exchangeable pressure cylinder in the device according to the invention, and there is also no pump with motor, the weight of the device is low compared to the prior art. This relatively low weight of less than 6 kg increases the patient mobility. The ease of use for the user, patient and nursing or care staff is also high. The wound drainage device according to the present invention can be used independently of a central vacuum system. Therefore, the wound drainage device according to the invention provides the patient with a high level of mobility. By way of example, a patient who has been operated on can be connected to the wound drainage system according to the invention in the operating theatre and can then be moved via the recovery room to the nursing area, during which process the wound drainage device can continue to function continuously. The wound drainage system according to the invention is also suitable for applications in home care, on account of its ease of use for patient and operating staff. A mobile patient can, for example, wear the wound drainage device according to the invention on a belt or move it around with him hanging from a mobile stand. The system can also be used at the bedside with the aid of suitable attachment means.

The wound drainage device according to the invention is in principle completely mechanical. An electricity source, whether external or internal, is not required, and consequently the wound drainage device according to the invention is safe despite the presence of moisture during use. The wound drainage device according to the invention uses gas-transformer means, in particular what is known as a compressed-air transformer, for generating vacuum with the aid of pressurized gas. Gas-transformer means of this type are commercially available. Examples thereof comprise a Venturi tube and piston-cylinder assemblies. With a view to noise reduction a Venturi tube is preferred, because it does not comprise any moving parts contrary to piston-cylinder assemblies. It should be noted that the use of a compressed-air transformer per se has already been proposed in NL-C-1006001.

To keep the reduced pressure exerted on the wound constant (for example at 140 cm H₂O), the current pressure is measured and a control signal based on prior calibration is emitted to the gas-transformer means.

The ability of the gas compartment to withstand pressure (maximum pressure is about 15 bar) does not impose any additional requirements on the materials used for the device. Standard plastic materials are used advantageously, as they yield upon excess load and therefore the risk of explosion is low. Furthermore, plastic materials contribute to the relatively low total weight of the entire device. The device according to the invention may comprise one or more gas compartments. The gas compartments may be connected in parallel or in series with respect to one another and can be connected to the gas-transformer means.

The transformer means can advantageously also be connected directly to an external gas source, so that in the event of a gas compartment not functioning, for example in the event of a leak or other defect, the wound drainage device can still be used and there is no need to interrupt the suction of exudate. In a preferred embodiment, to this end the coupling can be selectively connected to a gas compartment or the transformer means. By way of example, for this purpose the coupling is in communication, via a passage, with a gas compartment, which passage is provided with a side passage which is in communication with the gas-transformer means, the passage being provided with a shut-off member, such as a three-way valve. An additional advantage of this feature is that the contents of a gas compartment can be saved if the device is connected to a central gas source.

If desired, the compressed-air transformer may be suitable to be set to various vacuum pressure ranges, for a low vacuum, a medium vacuum and a high vacuum. The system selected by a treating physician to discharge the exudate is partly dependent on the wound which is to be treated. In the case of a high vacuum, the suction force is great, with the result that damage to the tissue may occur with certain types of wound. It is therefore advantageous if the reduced pressure in the collection pouch, and therefore the vacuum on the wound, can be set reproducibly and/or measured. For this purpose, the prior art has already disclosed pressure-monitoring and control systems for wound drainage devices, although these are generally only suitable for one specific type of pressure, whether high, low or medium. To prevent possible errors being made by the patient, this setting change can advantageously only be carried out by people authorized to do so. The wound drainage device can advantageously be set to one or more fixed reduced-pressure values, for example 150 cm water column for common wound drainage and 80 cm water column for retransfusion, such as by means of a control knob, and the device is calibrated in advance, so that when the reduced pressure is being measured a suitable control signal is fed to the gas-transformer means. To this end, the device is advantageously provided with a pressure gauge for measuring the reduced pressure. The combination of gas compartments and gas-transformer means as used in the wound drainage device according to the invention, is on its own not suitable for a thorax function, which requires a minimum flow velocity of about 20 l/min. Accordingly, in a preferred embodiment of the wound drainage device according to the invention a closable direct connection for coupling to a high vacuum system having a large flow is provided.

To allow a low consumption of the gas stock in the gas compartments, the gas-transformer means advantageously comprise two double-acting piston-cylinder assemblies, a working cylinder and a vacuum cylinder, the pistons of which are provided on a common piston rod. Displacement of the piston in the working cylinder under the influence of the pressure of the pressurized gas brings about a corresponding displacement of the piston in the vacuum cylinder and thereby generates vacuum in the vacuum cylinder on the reduced-pressure side thereof. When the direction of movement of the piston in the working cylinder is reversed, a vacuum is formed in the vacuum cylinder on the other side of the piston. The reduced-pressure side of the piston in the vacuum cylinder is in each case connected to a vacuum compartment, which in turn is in communication with the space between the receiving container and the wall of the vacuum chamber. The diameter of the vacuum cylinder is advantageously greater than, for example double, the diameter of the working cylinder, so that only a small amount of compressed air is required to generate a relatively high vacuum. The driving of the working cylinder is based on a control signal from the pressure gauge which measures the vacuum which is generated, so that the working cylinder can be operated discontinuously, in other words only when the measured vacuum is insufficient. This allows effective use of the gas stock in the gas compartments.

In an alternative embodiment a Venturi tube is applied as gas-transformer means, which tube generates less noise from its own compared to piston/cylinder assemblies. Furthermore this noise comprising a hissing sound from the exiting air can be reduced further in an efficient way by small noise absorbing dampers, for example made from gas permeable plastic foam, at the outlet of the Venturi tube.

In order not to have to clean and sterilize the entire wound drainage device in the event of damage to the collection container, for example a plastic receiving pouch, it is advantageous for a removable inner container to be arranged in the vacuum chamber and for the gas-transformer means to be in communication with the space between this inner container and the collection container.

The collection container is advantageously disposable and can be removed from the vacuum chamber. This means that there is no contact between the exudate and the remaining parts of the device, which is safe for staff and the device.

With a view to easily assembling, preferably the wound drainage device according to the invention has a modular configuration. Advantageously, the modular configuration comprises a two part housing, in particular a front part and a rear part of plastic, at least one gas compartment, a vacuum chamber in particular a vacuum cup, and a (plastic) supporting plate on which the gas-transformer means are mounted.

According to a further aspect, the invention relates to an assembly of a wound drainage device according to the invention and a collection container for collecting exudate originating from a wound, comprising a flexible receiving container which is in communication with a feed for conveying exudate from the wound to the receiving container.

In a preferred embodiment, the vacuum chamber is provided with an opening and the collection container comprises a cover for closing off the opening. In this embodiment, positioning the cover on the opening results in the collection container itself, such as a flexible receiving pouch, being held in a correct position in the vacuum chamber.

To ensure correct positioning of the cover, the cover advantageously comprises a closure rim, such that the cover can only be positioned on the opening of the vacuum chamber in a single way. The closure rim is preferably in the form of an ellipse composed of two ellipse parts of different eccentricity which adjoin one another. The opening is then provided with a closure rim of corresponding shape. The cover can be positioned on the vacuum chamber in one way only because of the different radii of the ellipse parts, from which the closure rim is composed. This ellipse shape is also advantageous since during drainage the receiving container, as seen in cross section, generally adopts the shape of an ellipse. The receiving container itself is made from flexible material in order to be deformed under the vacuum applied to the outer side of the receiving container, so that it can itself apply a vacuum to the wound. The vacuum chamber, advantageously a releasable cup in connection with the modular configuration, is therefore likewise preferably correspondingly elliptical in cross section. The vacuum chamber and/or collection container is advantageously provided with a scale marking indicating the volume of exudate which has been collected. Together, the closure rim and the cover form a vacuum-tight closure of the vacuum chamber. To enable the vacuum to be broken easily, it is advantageous for one or more lips to be provided on the cover. The underside of the cover is preferably provided with a number of reinforcing ribs in order to prevent the cover, which consists of flexible and relatively weak material with a view to forming a vacuum seal at the edge, from being sucked into the vacuum chamber under the influence of the vacuum. A balance between these contradictory requirements is achieved by providing the cover per se with a slightly convex top side (optionally achieved by means of the reinforcing ribs).

In a further embodiment, the cover is provided with a closable feed opening for supplying auxiliary substances, which feed opening is in communication with the receiving container. Auxiliary substances of this type may be required, for example, if the collected exudate is returned to the patient. Retransfusion of this nature is advantageous since it limits the risk of infection. The closable feed opening can also be used to take samples from the collected exudate in order to study these samples. A septum or similar seal is preferably provided in the additional feed opening. With a view to sterility a break away lid or similar protection is positioned over the feed opening. If desired, the collection container can be provided with a lid in order to allow for closing the feed opening again.

The feed line for carrying exudate from the drain to the receiving container is advantageously provided with a nonreturn valve in order to prevent collected exudate from flowing back to the wound, for example in the event of unexpected failure of the device.

With a view to retransfusion, the supply of exudate to the receiving container is preferably provided with a shut-off member, and the receiving container is provided with a discharge for removing exudate from the receiving container, which discharge is provided with a shut-off member. During drainage, the feed will be open and the discharge closed. For retransfusion, the feed is closed and the discharge opened. The feed and discharge are advantageously arranged on opposite sides of the receiving container, so that simple flow through the receiving container is possible.

Also with a view to retransfusion or other reuse of the collected exudate, in particular blood, the collection container is advantageously provided with a filter, in particular between the feed line and the receiving container. A filter of this type removes, for example, skin cells, blood clots and the like from the exudate supplied, which could otherwise block an injection needle used for retransfusion or could be unwanted otherwise. To prevent damage to blood cells, what is known as a sharp filter is not used, but instead thereof a filter with rounded openings. Since air is sometimes sucked in during drainage of a wound and likewise collects in the receiving container, thereby reducing the useful volume for exudate, the receiving container is advantageously provided with a filter which connects the inner side of the receiving container to the vacuum chamber during use, such that generating a vacuum to the wound is simple too. This ensures that the air which collects in the receiving container is removed by suction from the vacuum chamber through this filter. The filter is preferably of a type which swells when it comes into contact with moisture, closing up the pores in the filter, so that this filter is then fluid-tight.

The invention also relates to a collection container for collecting exudate originating from a wound, clearly intended for a wound drainage device and/or assembly according to the invention comprising a flexible receiving container, which is in communication with a feed for conveying exudate from the wound to the receiving container, and a cover, comprising a closure rim, such that the cover can be positioned on an opening of a vacuum chamber in a unique way. On account of this unique positioning, it is ensured that the collection container is put in place correctly. The closure rim is preferably in the form of an ellipse composed of two ellipse parts of different eccentricity which adjoin one another.

Furthermore, the invention relates to a collection container for collecting exudate originating from a wound, in particular in accordance with the embodiment of the invention comprising the cover described above, comprising a flexible receiving container which is in communication with a feed for conveying exudate from the wound to the receiving container, which feed is provided with a shut-off member, and a discharge for removing exudate from the receiving container, which discharge is provided with a shut-off member. This collection container according to the invention is particularly suitable for retransfusion, in which exudate which has been received in the receiving container during wound drainage is then reintroduced into a patient via the discharge, which for example is coupled to an infusion needle via a suitable line. By way of example, conventional valves can be used as the shut-off members.

The feed and discharge are preferably provided on opposite sides of the receiving container, in particular with the feed at the top side and the discharge at the underside, so that the receiving container can always be held in a virtually vertical position. To prevent a vacuum being sucked into the receiving container while the latter is being emptied, it is advantageous to provide a vent valve, preferably on the opposite side of the receiving container opposite to the discharge.

In a preferred embodiment of the collection container according to the invention, the collection container is provided with a filter, more preferably in the receiving container, in order to filter exudate which flows in.

The collection container is advantageously provided with a hanging eye, such as for example a hanging eye on the cover in accordance with the collection container of the invention described above. The beneficial features of the collection container discussed previously with respect to the assembly, are also applicable to the collection container per se.

The invention will be explained below with reference to the appended drawing, in which:

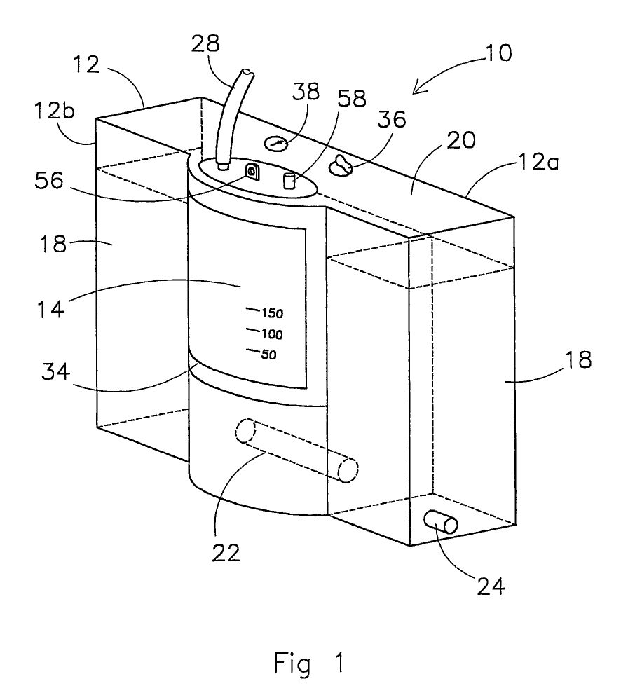

FIG. 1 shows a diagrammatic, perspective view of an embodiment of a wound drainage device according to the invention; and

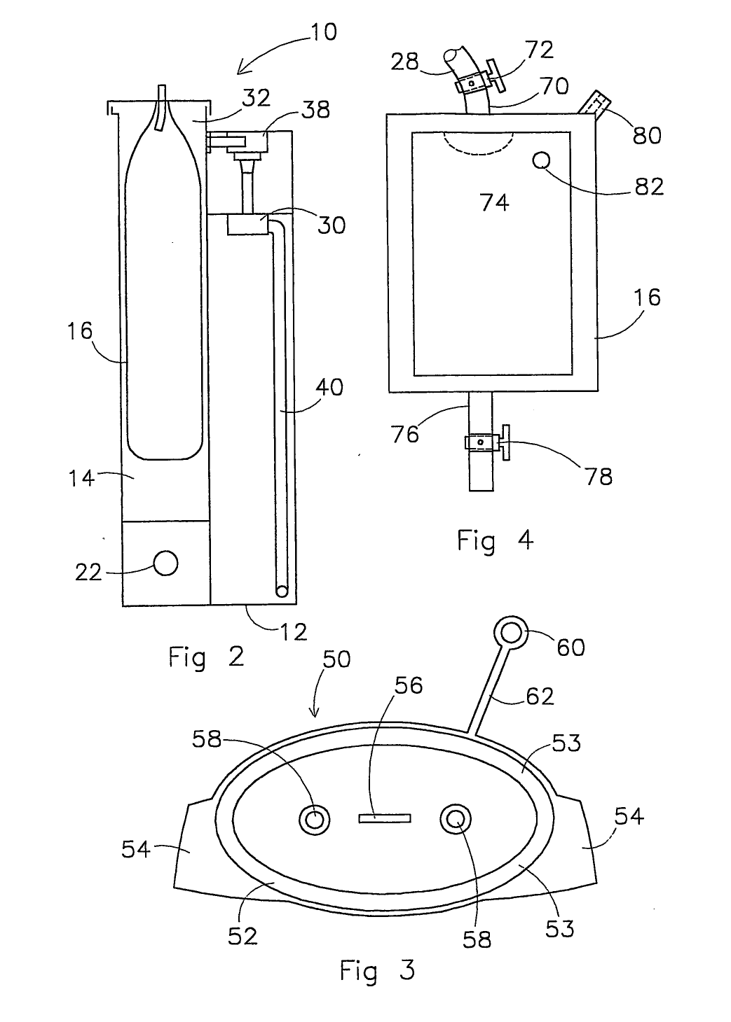

FIG. 2 shows a cross section through the device shown in FIG. 1;

FIG. 3 shows a plan view of a detail of an embodiment of a collection container according to the invention;

FIG. 4 shows a view of another embodiment of a collection container according to the invention; and

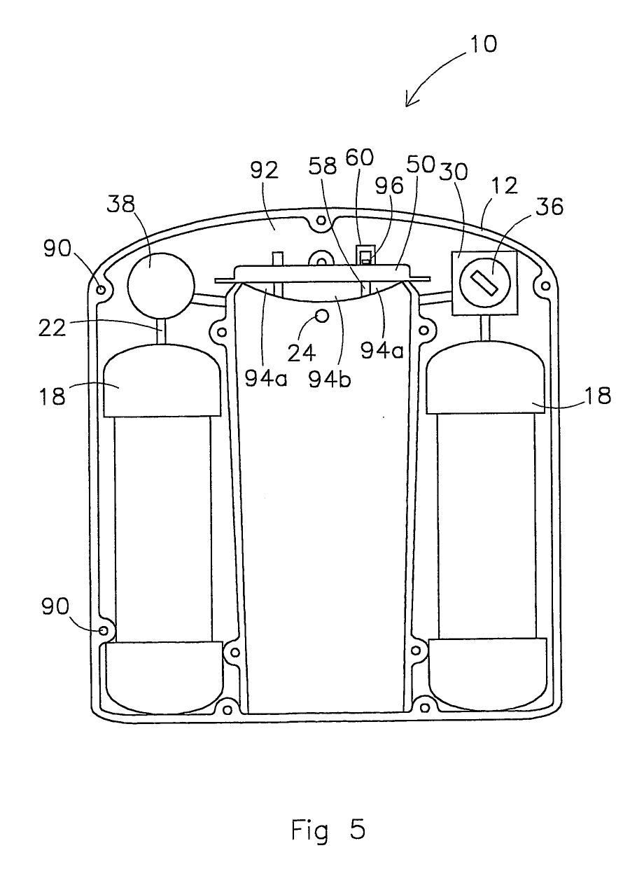

FIG. 5 shows a cut-away front view of another embodiment of a wound drainage device according to the invention; and

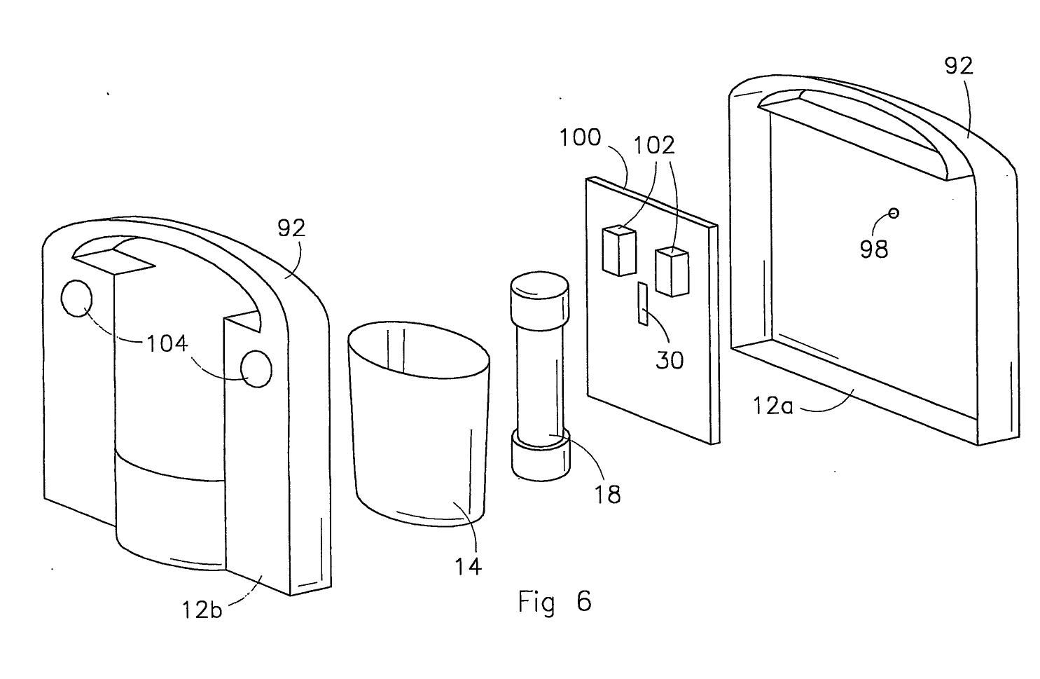

FIG. 6 shows a diagrammatic embodiment of the modular configuration of a wound drainage device according to the invention.

Corresponding parts are indicated by identical reference numbers in the various figures.

In the figures, the wound drainage device according to the invention is denoted overall by reference numeral 10. The device 10 comprises a two-part housing 12 made from plastic in which a number of compartments are provided. In this embodiment a vaccum chamber 14 forms an integral component of the housing 12 in which a removable flexible receiving pouch 16 (cf. FIG. 2) for exudate is accommodated. In other embodiments, the vacuum chamber is designed as a releasable cup. Pressure-resistant gas compartments that are releasable, and made from plastic, are arranged on either side of the vacuum chamber 14. At the rear side there is a connecting compartment 20, in which components which will be described in more detail below are arranged. In view of the relatively low pressure (up to 12 bar) which prevails in the gas compartments 18, it is sufficient for the gas compartments to be provided with a number of reinforcing ribs in order to achieve the required strength. In this embodiment, the gas compartments likewise form an integral component of the housing. This housing is, for example, injection-moulded in two parts, a front part 12a comprising all the compartments and a substantially planar rear part 12b. In this case, the gas compartments 18 are in communication with one another via a connecting passage 22. An external coupling 24 is provided for connecting the wound drainage device 10 to an external gas source, in particular a compressed-air line or compressor. The coupling 24 with shut-off member (not shown) is in communication with one of the gas compartments 18. One of the gas compartments 18 is connected at its top side to the pressure side of a compressed-air transformer 30, such as a Venturi-transformer, for generating vacuum with the aid of compressed air (or other pressurized gaseous medium). The suction or vacuum side of the compressed-air transformer 30 is in communication with the space 32 between receiving pouch 16 and the inner wall of the vacuum chamber 14. The receiving pouch 16 is in communication, via a suction line 28, with a drain (not shown) which can be fitted to a patient's wound. If desired, a disposable inner container (not shown) may be provided in the vacuum chamber 14 between the inner wall of the vacuum chamber 14 and the receiving pouch 16, which protects the vacuum chamber 14 in the event of any leak from or damage to the receiving pouch 16. The vacuum chamber 14 is provided with a window 34 with a scale marked on it, so that the liquid level in the receiving pouch 16 can be monitored from the outside. As an alternative to a scale being provided on the window 34, it is also possible for the receiving pouch to be provided with volume-indicating measurement marks for this purpose. In this embodiment, a switch 36 for operation of the device is provided on top of the device 10. The level of the vacuum can be read off from a manometer 38. Monitoring of the pressure value generates a control signal for the compressed-air transformer 30. The device also comprises a direct connection 40 between the external coupling 24 and the compressed-air transformer 30. FIG. 2 shows a cross section through the device 10.

FIG. 3 shows a plan view of a cover 50 for use with a disposable collection container according to the invention. The substantially flat cover 50 has a raised closure rim 52 having an inverted U shape in cross section, and which is elliptical in shape in view from above. The elliptical closure rim 52 is composed of two different ellipse parts 53 of different eccentricity. In other words, the closure rim describes a continuous curve. Lips 54 for taking hold of the cover 50 are provided at the longitudinal ends of the cover 50. A raised hanging eye 56 is located in the centre of the cover 50. Two holes 58 are provided in the longitudinal axis of the ellipse. One is for a flexible hose for connection to the actual drain and one is for the addition of auxiliary substances or for taking samples. The latter can be closed off with the aid of a cap 60, which cap is if desired secured to the cover 50 with the aid of a flexible link 62. The receiving pouch itself (not shown in this plan view) is secured to the underside of the cover 50 with the aid of suitable securing means, for example glue. If desired, a gasket can be provided.

FIG. 4 shows another embodiment of a collection container according to the invention. The collection container comprises a receiving pouch 16 made from a flexible material, which at the top side is provided with a feed 70 which, via valve 72, is in communication with a flexible hose 28 which is to be coupled to the drain. In this embodiment, a filter 74 being a mesh having round openings is arranged in the receiving pouch 16 directly below the feed 70 in order to filter the exudate flowing in. At the underside, the receiving container is provided with a discharge 76, which is likewise provided with a valve 78. A vent valve 80 is provided in a corner of the top side of the receiving container 16. An air permeable filter 82 is provided in the wall of the receiving pouch 16, through which air which is collected during drainage in the collection container 16 can be sucked out.

FIG. 5 shows a view of another embodiment of a wound drainage device according to the invention, from which a front part of the housing has been removed. Back part 12a of the housing comprises a number of threaded holes 90 for it to be secured to the front part with the aid of screws. For ease of handling, a handle 92 is integrated in the housing. In this embodiment, separate cylinder-shaped gas compartments 18, which are in communication with one another via line 22 and are also in communication with coupling 24, are arranged next to the vacuum chamber 14. In the case of the left-hand gas compartment 18, the line is in communication with manometer 38. Switch 36 and compressed-air transformer 30 are arranged at the right-hand gas compartment. As can be seen from this figure, the cover 50 is provided with reinforcing ribs on the underside. In each case two obliquely running, radial ribs 94a (only one of which can be seen in this view) extend from each hole 58 towards the closure rim. A further central reinforcing rib 94b is provided between the holes 58. A septum 96 is provided in the hole 58 at the right hand. This hole 58 at the right hand is provided with a breakable protective lid 60.

FIG. 6 shows diagrammatically an embodiment of a wound drainage device having a modular configuration. A plastic rear part 12a having an integrated handle 92 is provided with a direct connection 98 for coupling to an external vacuum system with a view to thorax function. A plastic mounting plate 100 can be mounted in the centre of the rear part 12a, e.g. by means of screws, after the components like pressure regulators 102 for the different ranges of reduced pressure and Venturi 30 have been pre-assembled on the mounting plate 100. For sake of simplicity not all the connections are shown. Subsequently, gas compartments 18 of plastic (only one shown) can be positioned at both sides of the mounting plate 100, and coupled to the further components, for example using flexible conduits of plastic. The vacuum cup 14 is positioned in the front part 12b of the housing, whereafter the front part 12b vacuum cup 14 is fixed to the rear part 12a, e.g. by screws. In this embodiment recesses 104 are provided in the front part 12b for incorporating switch 36 and manometer 38.

1. Wound drainage device for using reduced pressure: to discharge exudate originating from a wound, which device comprises a housing which comprises a vacuum chamber for receiving a collection container with a feed opening for collecting exudate, and means for generating a reduced pressure in the space between the inner wall of the vacuum chamber and a collection container which is accommodated therein during operation, wherein the said means for generating a reduced pressure comprise gas-transformer means for using pressurized gas to form a reduced pressure, which means on the pressure side are in communication with a pressure-resistant gas compartment and on the vacuum side are in communication with the vacuum chamber, the gas compartment being provided in the housing and having a coupling for connection to an external gas source.

2. Wound drainage device according to claim 1, wherein the coupling can be selectively connected to a gas compartment and the gas-transformer means.

3. Wound drainage device according to claim 1, wherein a removable inner container is arranged in the vacuum chamber, and the gas-transformer means are in communication with the space between the inner container and a collection container which is positioned in the inner container during operation.

4. Wound drainage device according to claim 1, wherein the wound drainage device comprises a modular configuration.

5. Wound drainage device according to claim 4, wherein the modular configuration comprises a two-part housing, at least one gas compartment, a vacuum chamber and a mounting plate having at least the gas-transformer means as components to be assembled.

6. Assembly of a wound drainage device for using reduced pressure to discharge exudate originating from a wound, which device comprises a housing which comprises a vacuum chamber for receiving a collection container with a feed opening for collecting exudate, and means for generating a reduced pressure in the space between the inner wall of the vacuum chamber and a collection container which is accommodated therein during operation, wherein the said means for generating a reduced pressure comprise gas-transformer means for using pressurized gas to form a reduced pressure, which means on the pressure side are in communication with a pressure-resistant gas compartment and on the vacuum side are in communication with the vacuum chamber, the gas compartment being provided in the housing and having a coupling for connection to an external gas source and a collection container for collecting exudate originating from a wound, comprising a flexible receiving container which is in communication with a feed for conveying exudate from the wound to the receiving container.

7. Assembly according to claim 6, wherein the vacuum chamber is provided with an opening, and the collection chamber comprises a cover for closing off the opening.

8. Assembly according to claim 7, wherein the cover comprises a closure rim, such that the cover can be positioned on the opening of the vacuum chamber in a unique way.

9. Assembly according to claim 8, wherein the closure rim is in the shape of an ellipse composed of two ellipse parts of different eccentricity which adjoin one another.

10. Assembly according to claim 7, wherein the cover is provided with a closable feed opening for supplying auxiliary substances, which feed opening is in communication with the receiving container.

11. Assembly according to claim 10, wherein the feed opening is provided with a septum.

12. Assembly according to claim 10, wherein a lid is provided for closing the feed opening again.

13. Assembly according to claim 10, wherein a breakable lid is provided for protecting the feed opening.

14. Assembly according to claim 6, wherein the feed is provided with a shut-off member, and the receiving container is provided with a discharge for removing exudate from the receiving container, which discharge is provided with a shut-off member.

15. Assembly according to claim 14, wherein the feed and discharge are provided on opposite sides of the receiving container.

16. Assembly according to claim 6, wherein the collection container comprises a filter.

17. Assembly according to claim 6, wherein the wall of the collection container comprises an air-permeable filter.

18. Collection container for collecting exudate originating from a wound, obviously intended for a wound drainage device for using reduced pressure to discharge exudate originating from a wound, which device comprises a housing which comprises a vacuum chamber for receiving a collection container with a feed opening for collecting exudate, and means for generating a reduced pressure in the space between the inner wall of the vacuum chamber and a collection container which is accommodated therein during operation, wherein the said means for generating a reduced pressure comprise gas-transformer means for using pressurized gas to form a reduced pressure, which means on the pressure side are in communication with a pressure-resistant gas compartment and on the vacuum side are in communication with the vacuum chamber, the gas compartment being provided in the housing and having a coupling for connection to an external gas source, the collection container comprising a flexible receiving container, which is in communication with a feed for conveying exudate from the wound to the receiving container, and a cover, comprising a closure rim, such that the cover can be positioned on an opening of a vacuum chamber in a unique way.

19. Collection container according to claim 18, wherein the closure rim is in the shape of an ellipse composed of two ellipse parts of different eccentricity which adjoin one another.

20. Collection container according to claim 18, wherein the feed is provided with a shut-off member, and a discharge for removing exudate from the receiving container, which discharge is provided with a shut-off member.

21. Collection container according to claim 20, wherein the feed and discharge are provided on opposite sides of the receiving container.

22. Collection container according to claim 18 wherein the collection container comprises a filter.

23. Collection container according to claim 18, wherein the wall of the collection container comprises an air-permeable filter.

24. Collection container according to claim 18 wherein the cover is provided with a closable feed opening for supplying auxiliaries, which feed opening is in communication with the receiving container.

25. Collection container according to claim 24, wherein a septum is provided in the feed opening.

26. Collection container according to claim 24, wherein a lid is provided for closing the feed opening again.

27. Collection container according to claim 24 wherein a breakable lid is provided for protecting the feed opening.
